(19) United States
(12) Patent Application Publication
(10) Pub. No.: US 2015/0150458 A1
(43) Pub. Date: Jun. 4, 2015

(54) IMAGING INTERFACES FOR FULL FINGER AND FULL HAND OPTICAL TOMOGRAPHY

(71) Applicant: The Trustees of Columbia University in the City of New York, New York, NY (US)

(72) Inventors: Andreas H. HIELSCHER, Brooklyn, NY (US); Christopher J. FONG, New York, NY (US)

(21) Appl. No.: 14/620,367

(22) Filed: Feb. 12, 2015

Related U.S. Application Data
(63) Continuation of application No. PCT/US13/54897, filed on Aug. 14, 2013.
(60) Provisional application No. 61/784,635, filed on Mar. 14, 2013.
(60) Provisional application No. 61/683,121, filed on Aug. 14, 2012.

(51) Int. Cl. A61B 5/00

(52) U.S. Cl. CPC A61B 5/0073; A61B 5/004

(57) ABSTRACT

Joint imaging devices methods and systems provide multiple sources and detectors around small accessible joints, such as fingers, to permit optical tomographic image acquisition and diagnosis of medical conditions such as rheumatoid arthritis. In embodiments, transducers permit convenient arrangement of sources and detectors and the rapid acquisition of anatomical surface geometry.

### CROSS-REFERENCE TO RELATED APPLICATIONS

The present application is a continuation of International Application No. PCT/US13/54897, filed on Aug. 14, 2013, which claims the benefit of U.S. Provisional Application No. 61/683,121, filed Aug. 14, 2012, and U.S. Provisional Application No. 61/784,635, filed Mar. 14, 2013, all of which are incorporated herein by reference in their entireties.

### FIELD

The present disclosure relates generally to medical imaging, and, more particularly, to optical tomography of human subject body parts, such as fingers and/or hands.

### BACKGROUND

Rheumatoid arthritis (RA) is a chronic, progressive, systemic, inflammatory disease that primarily attacks peripheral joints and surrounding tendons and ligaments. This disease, associated with significant pain and disability, affects about 1% of the population worldwide, and approximately 2.1 million people in the United States. While RA can be mild, 10% of affected subjects suffer total disability. In most human subjects, hand and fingers are the first body parts affected by this disease. Early detection of the disease may thus be possible by investigation of a human subject's hands and fingers.

### SUMMARY

Joint imaging devices methods and systems provide multiple sources and detectors around small accessible joints, such as fingers, to permit optical tomographic image acquisition and diagnosis of medical conditions such as rheumatoid arthritis. In embodiments, transducers permit convenient arrangement of sources and detectors and the rapid acquisition of anatomical surface geometry.

### BRIEF DESCRIPTION OF DRAWINGS

Embodiments will hereinafter be described with reference to the accompanying drawings, which have not necessarily been drawn to scale. Where applicable, some features may not be illustrated to assist in the illustration and description of underlying features.

FIG. 1A illustrates an optical tomography device for imaging a cross-section of a finger according to the prior art.

FIG. 1B illustrates an optical tomography apparatus for imaging a cross-section of a body part such as a finger using multiple points of illumination, according to one or more embodiments of the disclosed subject matter.

FIGS. 2-4 illustrate an optical tomography apparatus with various features that may be used alone or combined to position cameras in selected or various positions and orientations with respect to a target body structure and to position light sources in selected or various positions and orientations, according to one or more embodiments of the disclosed subject matter.

FIG. 5 shows an optical tomography apparatus in which a multiple points of illumination are formed using multiple contact-type light sources which are selected by an optical switch and in which one or more cameras may be used to detect transmitted light, according to one or more embodiments of the disclosed subject matter.

FIGS. 6 and 7 illustrate a an optical tomography apparatus in which a multiple points of illumination are formed using multiple non-contact-type light sources which are selected by an optical switch and in which one or more cameras may be used to detect transmitted light and in which the cameras and/or light sources may be translated to selected positions or to capture images of multiple zones of a body part, according to one or more embodiments of the disclosed subject matter.

FIG. 8 shows an optical tomography apparatus with a controller, a fixed camera capable of imaging a range of locations of the skin of a body part and an illumination source that generates multiple sources on the skin of a body part, according to one or more embodiments of the disclosed subject matter.

FIG. 9 shows an optical tomography apparatus with a controller, movable camera capable of imaging respective ranges of locations of the skin of a body part and a movable illumination source that generates multiple sources on the skin of a part by translating with respect to the body part, according to one or more embodiments of the disclosed subject matter.

FIG. 10 shows an optical tomography apparatus with a controller, a movable camera capable of imaging respective ranges of locations of the skin of a body part and one or more fixed illumination sources that generate(s) multiple sources on the skin of a body part, according to one or more embodiments of the disclosed subject matter.

FIG. 11 shows an optical tomography apparatus with a controller, multiple cameras in respective positions and orientations for imaging respective ranges of locations of the skin of a body part and a movable illumination source that generate(s) multiple sources on the skin of a body part, according to one or more embodiments of the disclosed subject matter.

FIG. 12 shows an optical tomography apparatus with a controller, multiple cameras in respective positions and orientations for imaging respective ranges of locations of the skin of a body part and a movable illumination source that generate(s) multiple sources on the skin of a body part at each selectable position acquired thereby, according to one or more embodiments of the disclosed subject matter.

FIG. 13 illustrates features of a support for a hand to prevent cross-illumination and to help positioning of the hand, according to one or more embodiments of the disclosed subject matter.

FIG. 14 illustrates a variation of a support for a hand in which openings giving a view to one or more cameras below the support are provided, according to one or more embodiments of the disclosed subject matter.

FIGS. 15A and 15B illustrate a glove transducer of resilient material that has markers for acquiring the surface shape of the target anatomy and supports sources and detectors at joint locations to position the latter when the glove transducer is worn by a person, according to embodiments of the disclosed subject matter.

FIGS. 16A through 16F illustrate a clip transducer that can be attached to a finger, according to embodiments of the disclosed subject matter.

FIGS. 16G and 16H illustrate a variation of the clip transducer of FIGS. 16A-16F with FIG. 16G showing the clip partially open and FIG. 16H showing the clip closed, according to embodiments of the disclosed subject matter.

FIGS. 17A, 17B, and 17D illustrate a clamshell or box transducer that the user can slip his hand into which has a resilient pocket to position sources and detectors in contact with the skin of a user's body, such as a hand, according to embodiments of the disclosed subject matter.

FIGS. 17C and 17E illustrate an inflatable transducer that the user can slip his hand into which has a resilient pocket to position sources and detectors in contact with the skin of a user's body, such as a hand, according to embodiments of the disclosed subject matter.

FIGS. 18A and 18B illustrate a strap-type transducer that can be wrapped around the user's joint according to embodiments of the disclosed subject matter.

FIGS. 19A and 19B illustrate a finger cot-type transducer that can be wrapped around the user's joint according to embodiments of the disclosed subject matter.

### DETAILED DESCRIPTION

Finger imaging apparatus can make use of optical tomographic imaging techniques, for example, to assist in the diagnosis of RA. Such an approach is described in International Publication No. WO 2012/082789, entitled “Medical Imaging Devices, Methods, and Apparatus,” which is hereby incorporated by reference herein in its entirety. Using an approach as described in International Publication No. WO 2012/082789, a finger of a human subject can be illuminated with light (e.g., near infrared (NIR)), and transmitted light intensities can be measured. For example, a laser can illuminate a point on a joint such as a finger joint which is immobilized or resting on a transparent plate. The measurement signal is converted to data and then used in an image reconstruction algorithm as described in International Publication No. WO 2012/082789 to generate three- and two-dimensional cross sections of optical properties throughout the finger and/or applied to a machine classifier to identify diagnostic features. With an appropriate classification algorithm, sensitivities and specificities of over 90% in diagnosing RA are possible. Note that generation of images may be a stage in the machine-classification process.

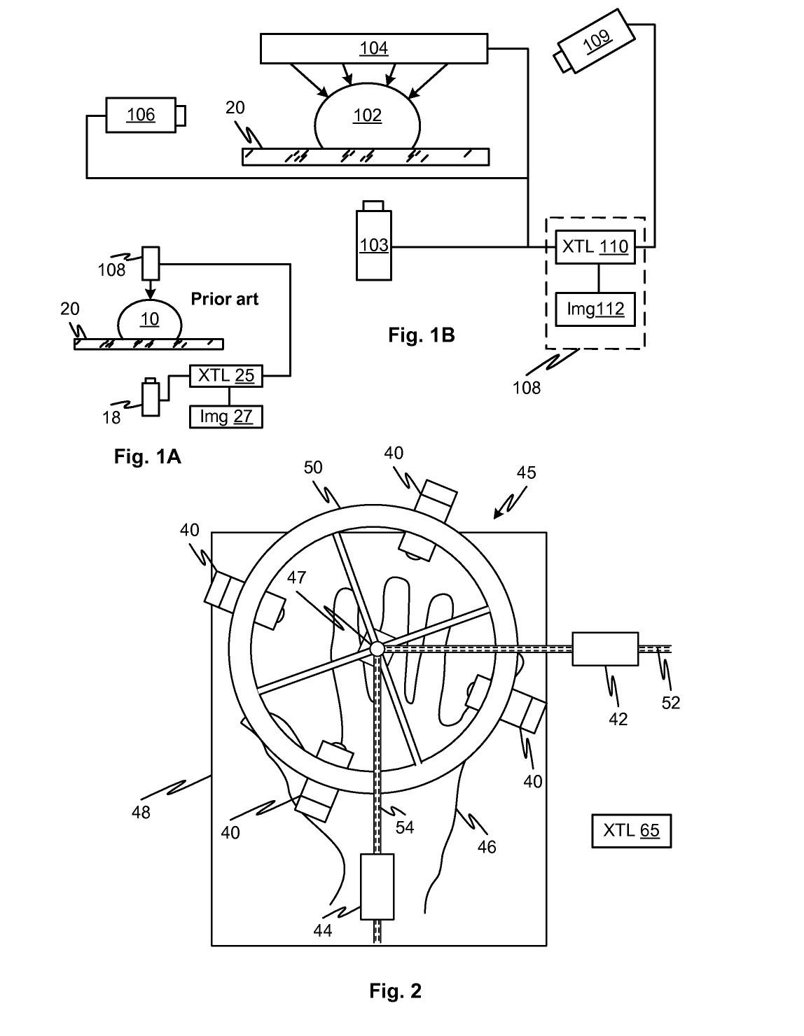

An example of an optical tomography apparatus of the prior art which is used for imaging a portion of a human subject's finger is shown in FIG. 1A. The apparatus uses a single light source 108, for example a laser that generates an optical source at one location into the back of the finger 10. The transmitted light intensity is detected by a camera 18 (e.g., a CCD camera) on the other side of the finger, which rests on a transparent support 20, such as a glass plate. The laser 108 can be scanned along a line on the back of the finger, for example, to be able to image multiple portions of the finger. A controller 25 may control the light source 108 and receive and digitize images of the transmitted and scattered light from the finger 10. The digitized image data may be provided to an image reconstruction processor of a host or embedded computer 27 and output on a display. Such a configuration, however, may only be capable of collecting limited imaging data for the reconstruction of optical properties inside the finger due to the single point of illumination per cross-section.

Note that in the present specification, the term “optical source” is used to denote the illumination boundary condition associated with the solution of optical tomography process and it refers to the physical effect of injecting light at a portion of a surface of the body part. The projection of light, such as a laser spot on the surface may be said to generate or create an optical source. If a laser spot is moved among multiple positions, it may be said that multiple optical sources are provided. The same is true if multiple laser spots are provided that are simultaneously projected on different locations. These would be respective sources provided at different times. The benefit of having multiple sources is increased information content so that it will be understood that the distribution of light on the surface and emitted from the body part for each of multiple sources is combined to generate an estimation of the distribution of at least one optical property within the body part. When a camera is used, each pixel can serve as a detector of a respective part of the surface of the body part. These are effectively separate data channels and are often referred to in the optical tomography art as “detectors.” So a camera image can provide multiple detectors, up to one for each pixel of the image, although pixels may be combined to enhance the intensity resolution at the expense of the spatial resolution. Detectors can also be discrete light sensors, each receiving light from a predefined region of the surface of the body part. Such detectors can be non-imaging, for example, ones that are in contact with the surface. Thus, the declaration in the present specification that optical sources are generated refers generally to the illumination of a discrete portion of the surface of a body part at a particular brief or extended period of time. The provision of multiple detectors refers to discrete detectors or to one or more images obtained by an imaging light sensor such as a camera. In the present specification, cameras may also be used for acquiring surface geometry as discussed elsewhere herein.

In one or more embodiments of the disclosed subject matter, multiple optical sources are generated on the surface of a body part and transmitted and scattered light is imaged at multiple points, effectively providing multiple detectors (each pixel being, potentially, an independent detector). This increases the amount of imaging data for the reconstruction of optical properties inside a portion of the human subject's body, such as a finger. The points of illumination may be generated sequentially so that at a time t₁, a first source is generated, then at time t₂, a second source is generated, and so on for the n sources up to tₙ. At each instant, the optical source may be frequency or amplitude-modulated as known in the art. The additional imaging data provided by detection from the multiple sources and the multiple detectors improves the quality of the cross-section and further assist in diagnosis of medical conditions. In embodiments, the optical tomographic data is used to diagnose arthritis. In embodiments, the sources are generated on a surface of a joint.

For example, as shown in FIG. 1B, an illumination apparatus 104 can provide for multiple points of illumination on a portion of a human subject body part 102 thereby defining multiple sources on the surface of the body part. The illumination of the successive points may be sequential such that only a single source on a surface of the body part 102 exists for each of multiple instances of time. The illumination apparatus may achieve the multiple points of illumination by switching a light source between multiple optical paths (e.g., using an optical switch to direct light from the source to one of a number of fixed waveguides), by providing multiple individual light sources (e.g., individually addressable laser or other light sources), by physically moving a light source between different locations (e.g., by using a translation and/or rotation stage), or by any other means (e.g., using tilting mirrors to redirect the light along different optical paths in free space).

A detection apparatus including one or more cameras, such as camera 103, 106, can be provided on an opposite side of the portion 102 or to the side, or viewing the body part 102 from one or more selected angles to detect light emanating from the body part 102 as a result of the illumination. For example, the cameras 103, 106 may include a photodetector (e.g., CCD camera) with a sufficiently large detectable area so as to detect light from the portion 102 regardless of the position of illumination. Alternatively or additionally, the cameras can include can include one or more optical components (such as a lens) for directing light from the body part 102 to the photodetector. A CCD camera may be used for each of the identified cameras. Intermediate optics may be used, for example, one or more optical fibers can be disposed for collecting light from the body part 102 and conveying it to a remote photodetector (not shown).

A further imaging device such as a camera 109 or laser scanner-based 3D-imager may be used for acquiring a surface geometry of the body part 102. The surface geometry may be used to configure a configurable illumination apparatus 104 with aimable lasers by providing a controller that can estimate features of the geometry such as a joint and position the optical sources responsively.

A control apparatus 308 can include a control module 310 that is coupled to the illumination apparatus 104 and/or the cameras 103 and 106 for controlling the operation thereof. The detection apparatus 106 can generate signals indicative of the amount of light detected for a given illumination position and can convey these to the control module 110. An imaging module 312 can use the generated signals from the cameras 103 and 106 to generate one or more images of the portion of the human subject. For example, the imaging module 312 can generate a two-dimensional cross-sectional image of the portion 102, which may be the finger of a human subject. The imaging module 312 can also be constructed to generate a three-dimensional image of the portion 102 by combining multiple two-dimensional cross-sections taken at different locations along an axis of the portion 102.

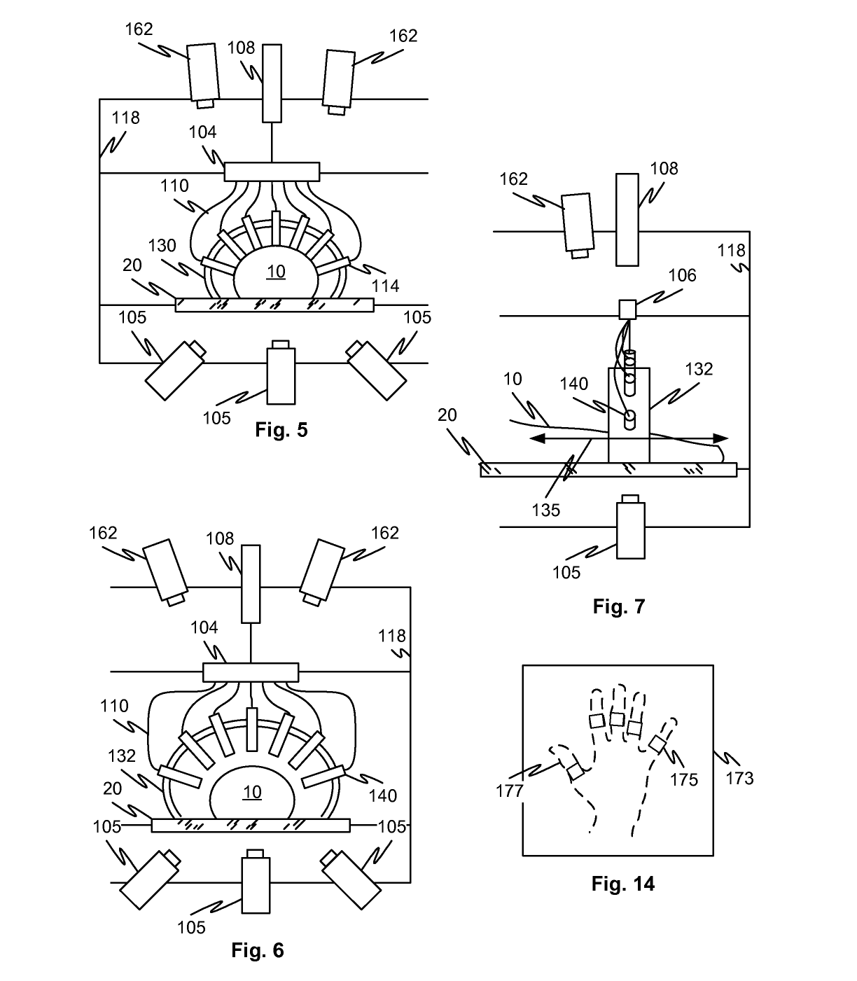

Referring now to FIG. 5, additional light illumination points and corresponding sources may be formed using contact-type illumination devices 114 supported by a support 130 and connected, respectively, to the ends of optical fibers 110 and contact the skin of a body part 10. An optical switch may be used to guide light from the laser into selected optical fibers that illuminate the finger at seven different positions. Although seven optical fibers are shown, it is to be appreciated that any number of optical fibers and/or illumination points can also be used. For example, a greater numbers of optical sources can be used to increase the information content of the resulting image. In addition, in a method, the illumination devices 114 can be moved relative to the body part 10 to provide additional optical data to further increase the information content available for diagnosis. As in the previous embodiments, a controller and imaging computer may be provided as discussed above. An optical switch transfers light from a light source 108, such as a laser, selectively to the fibers 110 under control of a controller. A support 20 may be used to immobilize or stabilize the body part 10. Cameras 105 may be positioned at multiple locations and orientations in order to capture light from different parts of the surface of the body part 10 and emitted in different directions relative to the local surface portions of the body part 10.

A non-contact illumination embodiment, as shown in FIGS. 6 and 7, allows more convenient repositioning of the light sources and may permit automatic longitudinal traversal of the body part. In such an embodiment, the optical sources and, optionally the camera(s) 105 can be moved along an axis to provide selected positioning of the optical sources. In embodiments, this may be provided by a motor driven traversing apparatus under control of the controller so that multiple sources may be generated for each of multiple traversal apparatus positions for each of several illumination devices 140.

A light source 108 such as a laser is switched among different illumination devices 140, each of which focuses light onto the surface of a body part 10 to generate an optical source. The illumination devices 140 are supported by a support 132. The body part is supported, restrained, rested, or immobilized by a support 20 which may include a transparent plate through which light from the body part may pass. Cameras 105 may be positioned at various locations and angles to capture light from different parts of the surface of the body part 10 and emitted in different directions relative to the local surface portions of the body part 10.

Fibers 110 can be terminated with illumination devices 140 such as collimators or lenses that focus light to generate a source on the surface of the body part 10 without contacting it (e.g., less than, or approximately, 1 mm in diameter) on the surface of the skin. For each of the sources, transmission data is collected with the one or more cameras 105. The image data is further processed as described above and in International Publication No. WO 2012/082789 to reconstruct the optical properties inside the body part. The illumination devices 140 can be supported by a support 132. The support 132 may have an arcuate or semi-circular configuration, although other shapes and configurations are also possible according to one or more contemplated embodiments.

By employing a translation stage (not shown), selected portions connected to a translating chassis 118 may be moved. For example, the light source 108 and switch 104 may be positioned at selected one or more positions to generate a selected set of optical sources or multiple optical sources along an axis of translation. For example, the longitudinal axis indicated at 135 may be scanned. The one or more cameras 105 may also be moved or may remain fixed. For example the field of view of the cameras 105 may capture a range of different positions on the body part 10 along the translation axis. In this case, only illumination points (and the corresponding sources) may be moved. Also, a traversing apparatus may not be needed if the light source 108 has an integral scanning component such as a mirror. For example, a DLP mechanism may be used to generate one or more selected sources on the body part 10 surface. Thus, a variety of mechanisms may be employed to achieve selected sources and detectors (again the pixels of the imaged surface represent independent detectors). Such a specific region or range of regions of the body part 10 may be scanned in order to acquire additional images of different cross sections along a selected axis. The apparatus may be modified to provide translation along multiple axes as well which may suit the interrogation of other body parts such as a head or breast.

The apparatus features may be further extended by creating translation stages for each finger (and respective illumination and/or detection apparatus), and scanning all fingers of the human subject's hand simultaneously. These features may be provided by wide field of view cameras and/or scanning light sources, as also discussed.

Instead of scanning the setup, a stationary configuration can be employed, in which additional optical fibers are placed around other cross-sections of the finger. This interface for one finger could be extended to cover all fingers and even the entire hand. Thus, the apparatus can be provided with a large number of optical fibers connected to one (or more than one) light source. For example, the number of fibers may be on the order of tens or hundreds of different illumination fibers, although other numbers are also possible according to one or more contemplated embodiments. Each of the different illumination fibers can be addressed sequentially by the optical switch. On the detection side, a single camera can be used to detect the light transmitted through the hand. Alternatively, multiple detectors can be used to detect the light transmitted for each finger or separate portion of the hand.

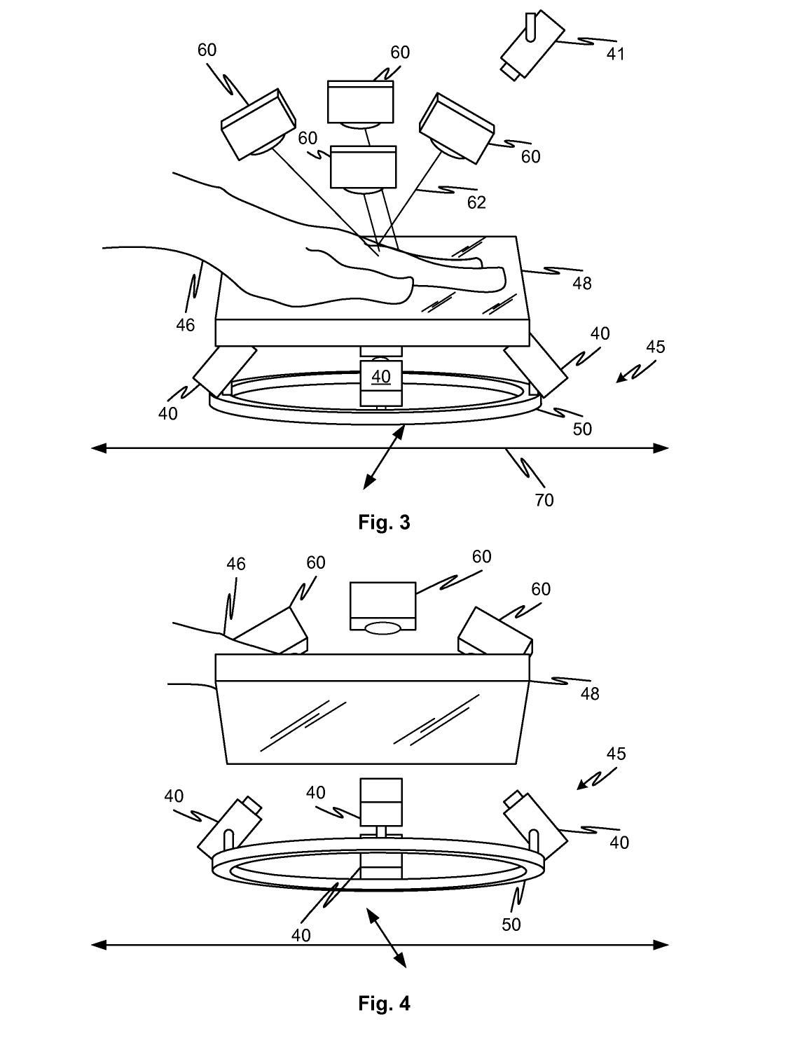

Referring now to FIGS. 2, 3 and 4, in other configurations, the photodetector (e.g., CCD camera) can be constructed such that the entire hand is in its field of view. As shown in the embodiment of FIGS. 2, 3, and 4, however, one or more cameras can be made movable and orientable by a motion apparatus 45 that may include a rotating gantry 50 and x and/or y translation stage 47. The translation stage 47, in the present embodiment, provides a stage that allows the gantry 50 to rotate therearound. The gantry 50 may be provided with an additional motor to permit cameras 40 to be positioned in various locations and with further motors to allow the camera 40 orientations to be altered. The translation stage 47 may be positioned by linear motors 42, 44 and shafts 52, 54, respectively. Although the apparatus for moving the translation stage 47 in two linear axes is shown, embodiments may provide for positioning and movement along a smaller or a greater number of linear axes. Additional rotational axes may also be added together to move the cameras 40 in different orientations and positions. All motors may be controlled by a controller 65. The human subject can place his/her hand 46 (dorsal side up) on the platform 48, which can be a transparent member such as a thin clear plastic or glass sheet provided between the illumination apparatus and the detection apparatus. Two circularly translating tracks, for example, about 2 feet in diameter, can be located above and below the platform and can have cameras 40 mounted at approximately a 45 degree angle to the center of the platform where the hand is placed. One or more cameras 41 can take pictures at multiple angles of the hand to be used to create a finite element mesh using appropriate software. The cameras 40 can be used for detecting the light emitted by the hand 46 as a result of one or more illumination sources 60 generated thereon by one or more light sources 60. The one or more light sources 60 may be arranged in a fixed position in or on a moving mechanism such as described with respect to the motion mechanism 67 of the cameras 40. Other devices or methods for creating such a mesh of the surface of the hand are also possible according to one or more contemplated embodiments. For example, a 3D scanner such as provided by the Microsoft Kinect interface may be employed.

As mentioned, a circular translational track can be a multi-directional translation stage, which can move one or more light sources, such as lasers, mounted thereon. This translation stage may be configured to move in an X-Y direction, rotate a gantry and also pivot at the base of each light source to orient each light source independently. Thus, a laser or lasers can illuminate the dorsal side of the hand of a human subject and can be translated so as to illuminate various points along the fingers, moved to various angles so that the angle with respect to the skin surface normal can be maintained within a desired range, and traversed longitudinally along fingers or to other joints such as a wrist or knuckle. With the ability to pivot or by means of the gantry or translation stage, the one or more light sources can also illuminate at multiple angles along the lateral parts of the fingers and wrists.

The finite element mesh can be acquired according to known methods. The mesh can be applied to a shape-feature recognition algorithm to allow the controller to guide the light sources and/or cameras to selected features. For example, the 3D mesh may indicate the geometry that includes several peaks along a continuous roughly cylindrical surface portion indicting the knuckles between phalanges. Optical sources may be generated at several locations across and axially from the peaks coinciding with the knuckles for imaging the important joints to diagnose arthritis.

As shown in FIG. 13, the transparent member 231 that the hand rests on may include depressions or fences 232 to hold the fingers apart and provide predictable and stable reference points for positioning the hand of a subject. In such a configuration, the human subject would only have to place their hand at the center of the platform with their fingers spread out at a comfortable distance during the imaging procedure. The fences 232 may be opaque to prevent cross-illumination of adjacent fingers thereby allowing multiple fingers to be scanned at the same time. For example, at each instant of time, first spots 251 and 252 on each of multiple fingers 261, 262 are generated. Then, at a second point in time, a second spot 253, 255 on each of the multiple fingers 261, 262 may be generated. The fence 258 separating the two fingers 261, 262 keeps the light from the sources going into adjacent fingers and reducing the image value captured by the camera. Other light blocking devices may be used, for example, an opaque glove with holes in it at the top and bottom faces would allow light to strike the skin and emanate from the palm side of the hand. Another feature that any of the disclosed embodiments may be modified to have is a support in which openings 175 are provided. FIG. 14 illustrates a variation of a support 173 for a hand 177, for example, in which the openings 175 permitting a view to one or more cameras below the support are provided. In respective embodiments employing such a support, the cameras that detect light emitted from the hand may be used for surface geometry acquisition as well as for obtaining the raw optical tomographic image data. Openings that are differently positioned, numbered, and shaped may be provided for the hand or for other body parts.

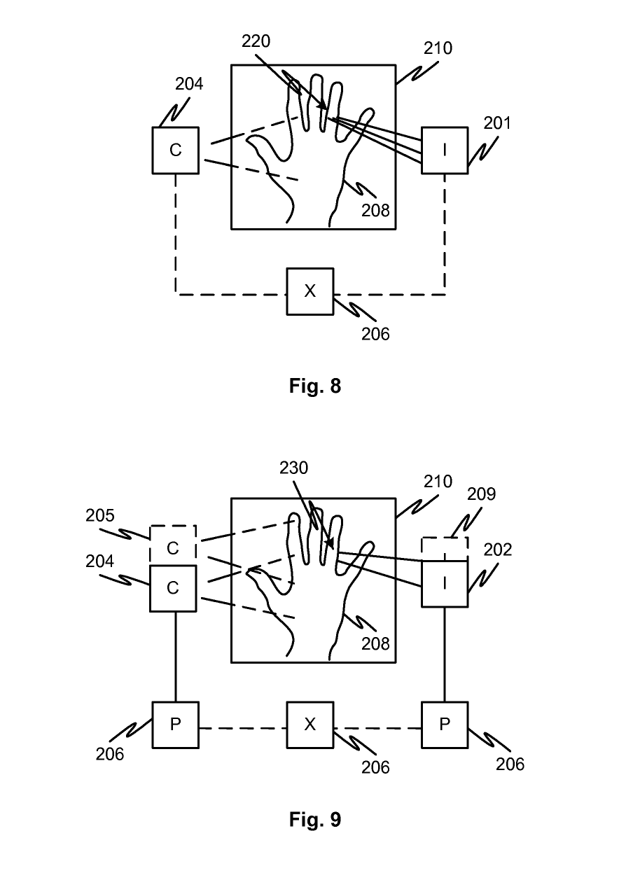

Referring to FIG. 8, an apparatus for performing diagnostic optical tomography on a hand 208 includes a support 210 for a hand 208 with a light source 201 configured to generate optical sources 220 at one or more points on the hand 208. A camera 204, which may be positioned on a side of the hand 208 opposite that of the light source 201, as illustrated in embodiments above, may have a field of view large enough to capture light from all parts of the hand 208 from which light emanates as a result of the sources. Thus the camera 204 can remain in a fixed position. The light source 201 can be configured to generate multiple optical sources 220 simultaneously or at respective points in time. The optical sources may be on different fingers, for example as discussed above in order to avoid having the light emitted from the hand and picked up the camera 204 including light from multiple optical sources at one time as discussed with regard to FIG. 13.

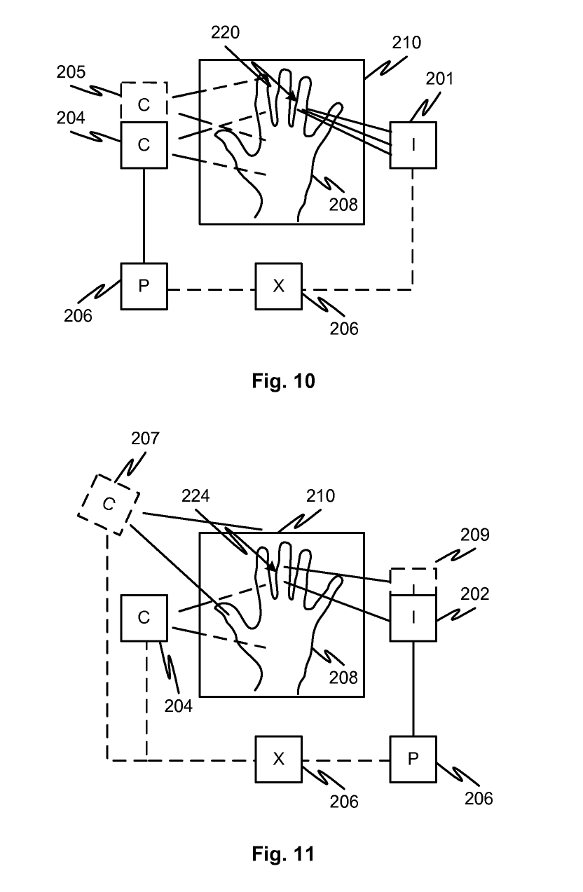

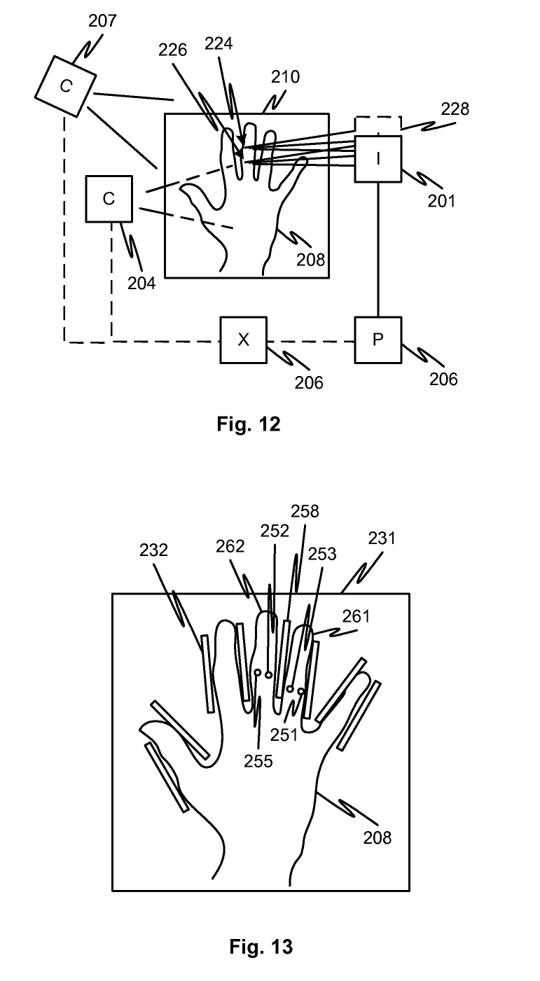

Referring to FIG. 9, apparatus for performing diagnostic optical tomography on a hand 208 includes a support 210 for a hand 208 with a light source 202 configured to generate optical sources 230 at one or more points on the hand 208. The light source 202 is movable to multiple positions (an additional example of which is shown at 209) by a positioning device 216 (examples of which are described above) controlled by controller 206. A camera 204, which may be positioned on a side of the hand 208 opposite that of the light source 201, as illustrated in embodiments above, may have a field of view large enough to capture light from all parts of the hand 208 from which light emanates as a result of the sources. The camera 204 may be moved by a positioning device 216 to change the orientation or position of the camera 204 to other positions such as illustrated at 205. The light source 202 can be configured to generate multiple optical sources 230 simultaneously or at respective points in time or a single source which is repositioned by the positioning device 216 or by a combination of these. For example, the apparatus of FIG. 10 is the same as that of FIG. 9 with the camera 204 as described with reference to FIG. 9. For another example, the camera's orientation may be changed as indicated at 205 by a motion apparatus or an additional camera 207 may be provided as shown in FIG. 11. FIG. 11 also shows a movable light source feature as in FIG. 9 combined with multiple cameras with respective positions and orientations. For yet a further example, FIG. 12 shows the multiple camera configuration referenced with regard to FIG. 11 and a movable, multiple-spot light source 201 (repositionable or re-aimable as indicated at 228) described with reference to FIG. 8. Note that the multiple-spot light source may be an optical switch and multiple optics or collimators as described with reference to FIGS. 1B, 5, 6, and 7 or it may be a single device such as a digital light processing (DLP) or microelectromechanical systems (MEMS) mirror-based device. Optical devices used by bar code scanners which scan laser beams using spinning mirrors or MEMS mirrors are suitable basis for a commercial device and may simplify the system.

FIG. 15A illustrates a glove transducer 300 of resilient material 304 such as latex or lycra. In alternative embodiments, the glove transducer 304 can be of mixed composition and made only to partly cover the hand but provide resilient support near joints to be inspected, for example, the finger joint areas indicated at 303 and 305. Markers 309 or other pattern provides an optical property that allows the position and angles of the surface of the glove transducer 300 to be acquired by one or more cameras 311. The image acquired by a camera, by capturing the planar projection of the markers 309 or other pattern can determine the slope. Alternatively, or in addition, by capturing images at multiple angles or by employing a laser scanner mechanism such as used by the Kinect video game interface, the surface geometry can be acquired. The markers 309 are not essential for acquiring the surface image since laser surface scanners do not require them. Other surface-geometry acquisition techniques may be employed as well.

The glove transducer 300 supports sources and sinks, one of which is indicated at 302. These may be powered by and signally connected to a terminal 314 by a conductor or signal cable 316. The terminal may also provide signal connection, for example wireless, to a controller 323. The controller 323 may also be connected by a communications cable (not shown) to the terminal 314. The terminal 314 may provide signal conditioning and data reduction to reduce the bandwidth of wireless communication to the controller 323. For example, the terminal 314 may handle the signal demodulation component of the imaging process and transmit the result to the controller 323 which may perform final processing or storage of the resulting data. The process may be as described in the International Publication WO 2013/049677 to Hielscher, et al, entitled: “Compact Wireless Optical Imaging Devices, Systems, and Methods,” hereby incorporated by reference in its entirety herein. In alternative embodiments, the terminal 314 includes source and detector components which transmit and receive light through fiber optic light guides to emit or receive light at the sources and detectors 302. In this case, the fiber optic light guides may be attached to the glove transducer 304 as also indicated at 316.

The glove transducer 300 may be provided with a specular reflecting surface, based on the technique used, to enhance surface geometry acquisition in addition to, or alternatively to, markers 309. The glove transducer 300 any pattern that is conducive to acquiring the surface shape of the target anatomy. The material may be a woven or nonwoven fabric or monolithic material that can hold the sources and detectors 302 against the skin of the wearer. The glove transducer 300 may also lack fingertips and/or have other openings such as in the palm and back of hand to make it more comfortable and easier to position and accommodate a wider range of sizes and anatomical features. For example, in embodiments, the glove may be shaped as a glove without at least one, several, or all of the fingers thereof being closed at a fingertip thereof. Although the sources and detectors 302 are shown arranged around three finger joints, they may be arranged at various other locations as well, for example at both the first and second finger joints, over the knuckles of the hand, over the wrist, over the thumb and fifth finger joints, etc. The sources and detectors 302 may have narrow waists to permit them to be fitted into elastic grommets 332 secured in suitable positions in the fabric 335 of the glove transducer 300 as shown in FIG. 15B. The elastic grommets 332 may be located at multiple positions to permit the sources and detectors 302 to be arranged to suit the particular conditions or anatomy to be inspected. The sources and sinks 302 may have conductors to carry power and/or signal data to the terminal 314. The

Note that instead of a glove, the features described above may be embodied in a shape for fitment over another body part such a foot or arm. For example, a sock transducer may be provided as a variant of the foregoing embodiment. An arm sleeve may form another embodiment.

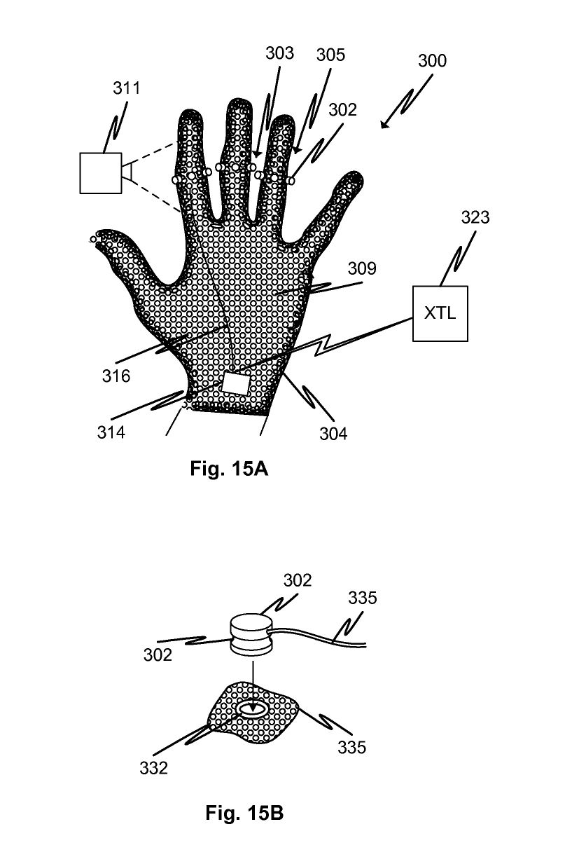

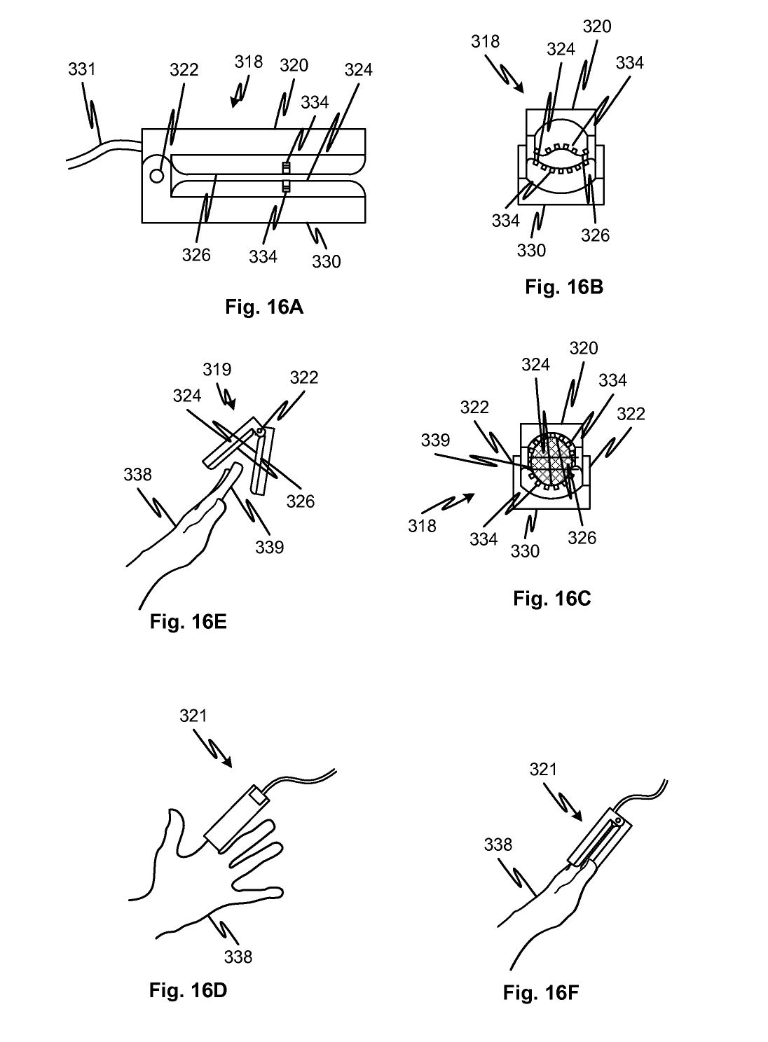

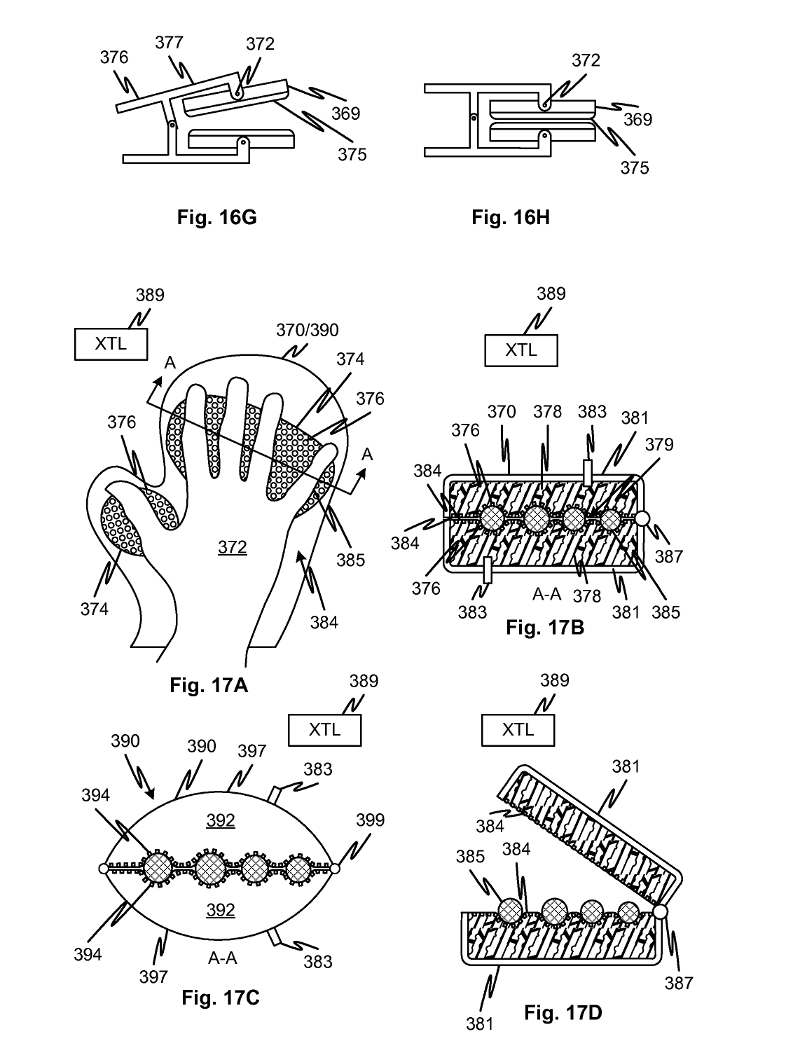

FIGS. 16A through 16F illustrate a clip transducer 318 that can be attached to a finger, according to embodiments of the disclosed subject matter. The clip may open as indicated at 319 in scissor fashion (See FIG. 16E) and then close under the urging of a spring (not shown) or other motor to clamp around a body part such as a finger 339 of a hand 338 as indicated in FIGS. 16D and 16F at 321 (FIGS. 16D and 16F show the closed clip around a finger from different perspectives.) Two parts may be joined by a hinge 322 to permit the opening and closing of the clip 318. Multiple clips 318 may be joined on a common chassis to permit multiple fingers to be inspected at one time. In such an embodiment, the clips may be opened and closed as a unit.

When the jaws 320 and 330 close around a finger, pillows 324 and 326 wrap sources and detectors 334 around the body part 339 to be inspected, in this case the finger 339. The pillows 324 and 326 may have sources and detectors 334 embedded in a closed cell foam cover or adhesively bonded to the pillows 324 and 326. The pillows may be filled with a fluid or a resilient foam. Alternatively they may be self-supporting convex surfaces without a backing which provide their own restoring force when forced inwardly due to the closing of the clip around a convex object such as a finger. In use, the clip 318 can be slid axially to align the sources and detectors 334 with the joint to be inspected. FIGS. 16G and 16H show a variation of the jaws 377 that allow the pillow 375 supports 369 to pivot around points 372 on the jaw 377 to allow a greater variation in finger shapes and widths to be accommodated. FIG. 16G shows the clip partially open and FIG. 16H shows the clip closed. Also shown are levers 376 which may be provided on this and other embodiments (e.g. 318, 319) to facilitate the opening of the jaws.

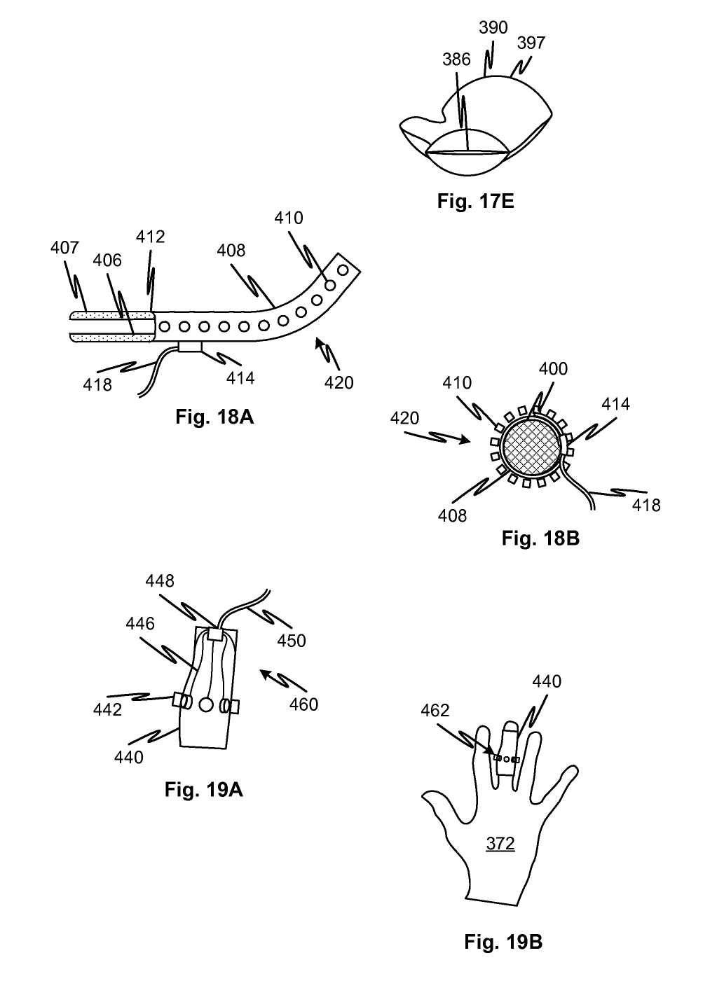

FIGS. 17A and 17B illustrate a clamshell or box transducer 370 into which a user can slip his hand 372 or other body part. The transducer 370 has a resilient pocket 379 lined by sources and detectors 376 which contact with the skin of a user's body, such as a hand 372. The transducer may have a shell 381 that is filled with a resilient material 378 such as a foam rubber or viscoelastic foam covered by a liner 384 carrying the sources and detectors 376. The sources and detectors may be arrayed over a region 374 of the liner 384 as can be seen in FIG. 17A so that they are positioned around joints to be inspected. In embodiments, only a subset of the sources and sinks 376 are used for inspection. The transducer may permit the selection of a subset based on

As shown in FIG. 17D, the shell may be opened, for example by pivoting about a hinge 387, to permit insertion of a hand. The open position may also permit identification of the positions of the joints to be inspected relative to the sources and sinks 376. The particular sources and sinks 376 to be used may be identified by inspection and instructions provided through a user interface of a controller 389 to activate only the identified ones of the sources and sinks 376. Alternatively, the controller can be programmed to identify the best sources and sinks 376 automatically by performing a preliminary image acquisition to identify the positions of the joints relative to the sources and sinks 376.

Instead of a resilient material 378, a fluid or air may fill the volume defined between the shell 381 and the liner 384. In this case, a port 383 may permit an operator to vary the amount of fluid in the volume. In embodiments, the shell 381 may be clamped shut and fluid filled through port 383 until a predefined pressure is generated.

FIG. 17C illustrates an inflatable transducer 390 that the user can slip his hand into which has a resilient pocket to position sources and detectors in contact with the skin of a user's body, such as a hand, according to embodiments of the disclosed subject matter. The inflatable transducer 390 may have sources and sinks 394 positioned and attached as described with reference to the previous embodiments with the primary difference being that there is no shell but rather one or two (or more) flexible bladders 397 which may be inflated using respective ports 383. In the illustrated example, two bladders 397 are connected, as shown in the cross-section view, along their edges as indicated at 399. The connection may be any suitable for forming a seam, such as an elongate clamp or stitching. Instead of opening like a clamshell as the embodiment of FIG. 17D, the bladders may form a configuration somewhat like a baseball glove as shown in FIG. 17E which has an opening 386 into which the hand may be inserted with the compliant interior walls carrying the sources and sinks flexing to accommodate. Note, the embodiments of FIGS. 17C and 17E may be configured as shown in FIG. 17A and described with reference thereto.

In alternative embodiments, a rigid member with sources and detectors may be positioned opposed to an inflatable bladder carrying sources and detectors and a body part, such as a hand, can be positioned between them. In this embodiment, the inflatable bladder may wrap around a substantial portion of the body part while the rigid member provides sources and detectors for the rest of the body part. In another example, one of the shells 381 may be combined with one of the inflatable bladders 397 which may be brought into opposition to achieve another alternative configuration.

FIGS. 18A and 18B illustrate a strap-type transducer 420 that can be wrapped around the person's joint. An elongate flexible member 408 may be of leather, woven or non-woven fabric, elastic material, composite or other suitable material. Sources and detectors 410 may be arrayed along the strap at multiple positions. Although a single row of sources and detectors 410 are shown, more than one row may be provided. The sources and detectors may be attached such that when wrapped around a joint 400, they transmit light into the joint 400 and receive light from the joint 400, respectively. The sources and sinks 410 may be powered, and signals applied and received through, conductive traces or leads attached to the elongate member 408. Alternatively, optical leads may be attached to the elongate member 408. The end of the elongate member 408 may have a slot 406 to allow the sources and sinks 410 to fit within so that attachment material 407 such a Velcro can contact an opposite side of the elongate member 408 to connect the ends thereof as shown in FIG. 18B. An electrical or optical cable 418 may provide for power and/or signal conduction.

FIGS. 19A and 19B illustrate a finger cot-type transducer 460 that can be wrapped around the user's joint 462 of a hand 372 or other body part. A cot 440 or flexible material carries multiple sources and sinks 442 arranged to surround the joint 440. The cot-type transducer 460 may be slipped over a finger and positioned over the joint 462 of interest. One or more cot-type transducers 460 may be position in this manner for a single imaging procedure. The cot 440 may carry wires or optical fibers 446 for carrying signals and/or power to the sources and detectors 442. A signal cable may conduct signals and/or power to/from a controller or a wireless connection may be provided.

In any of the foregoing embodiments in which a resilient of flexible material is laid over or urged onto the surface of a person's anatomy, at least aspects of the surface geometry may be acquired by directly detecting the shape of the surface. For example, the liner 384 may be provided with integral strain gauge or capacitive elements that allow its shape to be detected. Suitable technology has been used for creating glove-type input devices for computer. An example of a suitable technology can be found in U.S. Pat. No. 8,431,080 to Liu et al. In embodiments, the strain may be detected in a single direction, for example circumferential tension in a finger cot may indicate the diameter at a point. A controller may correlate the diameter with the locations of sources and detectors based on a look-up table and the signals from circular strain gauge tensionometers. Since the basic structure of a normal finger is known a priori, the limited additional information about the diameter may be sufficient for modeling the entire finger joint area.

In any of the embodiments described herein, the sources and detectors may be constituted by the ends of optical fibers with the electrical components positioned remotely from the surface points that are inspected. In such cases, the end may further include lens, prism, or diffusing structures to redirect and condition the light transmitted or captured, respectively.

Optical sources may be generated on different fingers, for example as discussed above, in order to avoid having the light emitted from the hand and picked up the camera 204 including light from multiple optical sources at one time as discussed with regard to FIG. 13.

In embodiments of the disclosed subject matter, a suitable human subject-instrument interface is provided for optical tomographic imaging of human subject appendages and/or tissues, for example, finger joints and hands, by providing multiple sequential illumination points around the tissue to be imaged. Waveguides and/or scanners are used to achieve such sequential illumination, but other methodologies for achieving the multiple points of illumination are also possible according to one or more contemplated embodiments. Embodiments of the disclosed subject matter can be used in the diagnosis of arthritis in human subject joints as well as in other applications. For example, similar interfaces can be used for imaging of other portions of the body, such as the feet or breasts. Applications of the methods and apparatus described herein are thus not limited to imaging of the fingers and hands alone. Configurations of the optical tomographic imaging apparatus as disclosed herein can increase the speed of data acquisition and the accuracy of image reconstruction, which thereby can result in faster and more accurate diagnosis.

Optical sources may be pulsed, time-harmonic, coherent or incoherent, multispectral or narrow or single-band. The detectors may be time or frequency-resolved. In embodiments, multiple laser spots can be projected on different parts of the hand with little or no cross-talk, which may allow the acceleration of the process of performing a diagnostic procedure. Opaque fences may be provided between fingers to facilitate this. Optical sources may be pulsed, time-harmonic, coherent or incoherent, multispectral or narrow or single-band.

According to first embodiments, the disclosed subject matter includes an optical tomography device for imaging at least one finger of a patient. A detection portion has a support configured to permit the hand of a person to rest thereon, the support being attached to a frame for an illuminating device configured to illuminate joint portions of the hand at a plurality of locations thereof. An imaging portion is configured to image light emanating from the hand of the person which results from the illumination at the plurality of the points and to generate image data representative of the light emanating from the hand, the points being surface locations and being at different positions on the surface of the hand. A processor is programmed to receive the image signals and combine the image data representing light emanating from the hand in response to each of the plurality of points. The processor is further programmed to estimate a distribution of an optical property within the hand by combining the image data corresponding to at least two of the plurality of points.

In variations thereof, further first embodiments may be formed in which the detection portion includes a photodetector or camera configured to detect and discriminate light emanating from each of a variety of points on the surface of the hand. In variations thereof, further first embodiments may be formed in which the illumination device includes a plurality of waveguides arranged such that each generates a respective one of the points. In variations thereof, further first embodiments may be formed in which the number of points is at least three. In variations thereof, further first embodiments may be formed in which the number of points is at least five. In variations thereof, further first embodiments may be formed in which the number of points is at least seven. In variations thereof, further first embodiments may be formed in which the number of waveguides is at least one hundred. In variations thereof, further first embodiments may be formed in which the waveguides are optical fibers. In variations thereof, further first embodiments may be formed in which the illumination device includes an arcuate or semi-circular holder for the output ends of the waveguides, the output ends being arranged at substantially equal intervals around the holder, the holder being sized to permit the insertion of a finger between the holder and the support. In variations thereof, further first embodiments may be formed that include an optical switch connected to input ends of the waveguides, and a light source connected to the optical switch, the switch being configured to selectively connect each waveguide to the light source. In variations thereof, further first embodiments may be formed in which the illuminating device includes a near-IR laser. In variations thereof, further first embodiments may be formed in which the output ends of the waveguides including collimators or lenses constructed to focus light passing therethrough. In variations thereof, further first embodiments may be formed in which the arrangement of the output ends with respect to the portion results in a spot size of the light from each waveguide incident on the surface of the portion of less than 1 mm in diameter. In variations thereof, further first embodiments may be formed in which the illumination device includes a plurality of individual light sources, each coupled to a respective input end of one of the plurality of waveguides. In variations thereof, further first embodiments may be formed that include a translation stage configured to move the illumination device to respective positions, each of which is effective to generate a respective one of the plurality of points. In variations thereof, further first embodiments may be formed in that include a translation stage configured to move the illumination device to respective positions, each of which is effective to generate a respective one of the plurality of points. In variations thereof, further first embodiments may be formed that include a translation stage configured to move the illumination device to respective positions, each of which is effective to generate a respective one of the plurality of points. In variations thereof, further first embodiments may be formed that include a plurality of the illumination devices, each of the illumination devices arranged to illuminate a different finger of the patient's hand. In variations thereof, further first embodiments may be formed that include a plurality of the illumination devices, each of the illumination devices being arranged to illuminate a different finger of the patient's hand. In variations thereof, further first embodiments may be formed that include a plurality of the illumination devices, each of the illumination devices being arranged to illuminate a different finger of the patient's hand. In variations thereof, further first embodiments may be formed that include a plurality of the illumination devices, each of the illumination devices being arranged to illuminate a different finger of the patient's hand.

According to second embodiments, the disclosed subject matter includes an optical tomography device for imaging at least one finger of a patient, the device. A support is sized and configured to permit the hand of a human subject to rest thereon. An illumination device is attached to the support. A surface geometry capture device is attached to the support. A light capture device is attached to the support and a controller is connected to the illumination device, the surface geometry capture device, and the capture device and configured to control each of them. The illumination device is configured to generate optical sources at multiple locations about a surface of a joint of a hand resting on the support. The light capture device is configured to provide multiple detectors on a surface about the joint of a hand resting on the support.

In variations thereof, further second embodiments may be formed in which the surface geometry capture device includes at least one camera oriented to image the dorsal surface of a hand. In variations thereof, further second embodiments may be formed in which the surface geometry capture device includes at least two cameras oriented to image the dorsal surface of a hand from at least two points of view. In variations thereof, further second embodiments may be formed further including a positioning device configured to move and/or orient the at least two or the at least one camera, wherein the controller is configured controls the positioning device. In variations thereof, further second embodiments may be formed in which the controller is configured to control the illumination device to generate multiple sources located at multiple locations along a lateral axis of the finger. In variations thereof, further second embodiments may be formed in which the controller is configured to control the illumination device to generate multiple sources located at multiple locations along a longitudinal axis of the finger. In variations thereof, further second embodiments may be formed in which the controller is configured to control the illumination device to generate multiple sources located at multiple locations along a longitudinal axis of the finger. In variations thereof, further second embodiments may be formed in which the controller is configured to provide multiple detectors on a surface about the joint of a finger resting on the support. In variations thereof, further second embodiments may be formed in which the light capture device is configured to provide multiple detectors on a surface about the joints of multiple fingers resting on the support. In variations thereof, further second embodiments may be formed in which the light capture device includes a CCD camera configured to provide multiple detectors on a surface about the joints of multiple fingers resting on the support. In variations thereof, further second embodiments may be formed in which the controller is configured to control the illumination device to generate multiple sources located at multiple locations along a lateral axis of each of the multiple fingers. In variations thereof, further second embodiments may be formed in which the controller is configured to control the illumination device to generate multiple sources located at multiple locations along a longitudinal axis of each of the multiple fingers.

According to third embodiments, the disclosed subject matter includes a method for acquiring a cross-sectional image of a body part of a human subject. The method includes illuminating multiple points on a surface of the body part at a desired cross-sectional plane. The method includes detecting light from the body part as a result of the illuminating and illuminating another point on the surface of the body part at the desired cross-sectional plane. The method further includes detecting light from the body part as a result thereof and generating the cross-sectional image of the body part of the human subject based on the light detected.

In variations thereof, further third embodiments may be formed in which the repeating the illuminating and generating are repeated at different cross-sectional planes. In variations thereof, further third embodiments may be formed in which the cross-sectional images are used to generate a three-dimensional cross-section of the body part. In variations thereof, further third embodiments may be formed in which the repeating includes translating a light source that provides the illuminating to a different location.

According to fourth embodiments, the disclosed subject matter includes an optical tomography device for imaging at least one finger of a patient, the device comprising. A support including an enclosure cover sized and configured to enclose the hand or finger of a human subject, the enclosure having a lining defining an inwardly-facing surface arranged to face, and contact, an enclosed hand, the sources and detectors being arrayed on the inwardly-facing surface. Light sources and detectors are attached to the surface and a controller is connected to the sources and detectors and configured to control them to generate an optical tomographic image. The sources and detectors are arranged to generate optical sources at multiple locations and optical detectors at multiple further locations about a surface of a joint of a hand of finger adjacent the surface. The light capture device is configured to provide multiple detectors on a surface about the joint of a hand or finger resting on the support.

In variations thereof, further fourth embodiments may be formed in which the lining includes sensor elements configured to output a signal indicating at least one geometric parameter of the surface. In variations thereof, further fourth embodiments may be formed in which the sensor elements include capacitors. In variations thereof, further fourth embodiments may be formed in which the sensor elements include strain gauges. In variations thereof, further fourth embodiments may be formed in which the support includes a finger cot with the sources and detectors supported thereby. In variations thereof, further fourth embodiments may be formed in which the surface is an internal surface of the finger cot. In variations thereof, further fourth embodiments may be formed in which the enclosure cover includes a rigid enclosure element that supports the lining which together encase a resilient foam member which resiliently supports the lining. In variations thereof, further fourth embodiments may be formed in which the enclosure cover is Tillable with a fluid. In variations thereof, further fourth embodiments may be formed in which the enclosure contains a bladder a portion of which includes the lining. In variations thereof, further fourth embodiments may be formed in which the enclosure is configured to wrap around a finger. In variations thereof, further fourth embodiments may be formed in which the enclosure is configured to clip onto a joint. In variations thereof, further fourth embodiments may be formed in which the enclosure includes two hingeably connected members each with a soft pillow, the lining forming the surfaces of each pillow, the hingeably connected members being urged together by an urging member to facilitate clamping onto a joint of a person. In variations thereof, further fourth embodiments may be formed in which the enclosure is shaped as a glove. In variations thereof, further fourth embodiments may be formed in which the enclosure is shaped as a sock. In variations thereof, further fourth embodiments may be formed in which the enclosure is shaped as a glove without fingertips. In variations thereof, further fourth embodiments may be formed in which the enclosure is shaped as a glove without at least one of the fingers thereof being closed at a fingertip thereof.

According to firth embodiments, the disclosed subject matter includes an optical tomography device for imaging a body part of a patient. An inflatable member supports multiple sources and/or detectors on an external surface thereof. A rigid member has sources and detectors. The inflatable member is connectable to the rigid member such that the respective sources and detectors of each face each other and face a body part placed between them. In variations thereof, further fifth embodiments may be formed in which the rigid member has a flexible resilient member with a lining thereover that carries the rigid member sources and detectors. In variations thereof, further fifth embodiments may be formed in which the resilient member includes a foam member. In variations thereof, further fifth embodiments may be formed in which the inflatable member and the rigid member are connected by a hinge. In variations thereof, further fifth embodiments may be formed in which the rigid member and inflatable member are sized and shaped to enclose the hand or finger of a human subject. In variations thereof, further fifth embodiments may be formed in which a controller is connected to the sources and detectors and configured to control them to generate an optical tomographic image. In variations thereof, further fifth embodiments may be formed in which the sources and detectors are arranged to generate optical sources at multiple locations and optical detectors at multiple further locations about a surface of a joint of a hand of finger adjacent said surface. In variations thereof, further fifth embodiments may be formed in which the surface of the inflatable member includes sensors configured to output a signal indicating at least one geometric parameter of said surface such that said signal is responsive to a shape of the body part. In variations thereof, further fifth embodiments may be formed in which the multiple locations and multiple further locations each number at least two. In variations thereof, further fifth embodiments may be formed in which the multiple locations and multiple further locations each number at least three. In variations thereof, further fifth embodiments may be formed in which the multiple locations and multiple further locations each number at least four. In variations thereof, further fifth embodiments may be formed in which the multiple locations and multiple further locations each number at least five. In variations thereof, further fifth embodiments may be formed in which the multiple locations and multiple further locations each number at least seven. In variations thereof, further fifth embodiments may be formed in which the multiple locations and multiple further locations each number at least two for joints of at least two fingers. In variations thereof, further fifth embodiments may be formed in which the multiple locations and multiple further locations each number at least three for each of at least one joint of at least two fingers. In variations thereof, further fifth embodiments may be formed in which the multiple locations and multiple further locations each number at least four for each of at least one joint of at least two fingers. In variations thereof, further fifth embodiments may be formed in which the multiple locations and multiple further locations each number at least five for each of at least one joint of at least two fingers. In variations thereof, further fifth embodiments may be formed in which the multiple locations and multiple further locations each number at least seven for each of at least one joint of at least two fingers. In variations thereof, further fifth embodiments may be formed in which the sources and detectors are located such that at least one source is displaced along a longitudinal axis from at least one detector. In variations thereof, further fifth embodiments may be formed in which the sources and detectors are located such that at least two sources are displaced along a longitudinal axis of a respective finger from at least two detectors.

It will be appreciated that the modules, processes, systems, and sections described above can be implemented in hardware, hardware programmed by software, software instruction stored on a non-transitory computer readable medium or a combination of the above. For example, a method for optical tomography can be implemented, for example, using a processor configured to execute a sequence of programmed instructions stored on a non-transitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C++, C#.net or the like. The instructions can also comprise code and data objects provided in accordance with, for example, the Visual Basic™ language, LabVIEW, or another structured or object-oriented programming language. The sequence of programmed instructions and data associated therewith can be stored in a non-transitory computer-readable medium such as a computer memory or storage device which may be any suitable memory apparatus, such as, but not limited to read-only memory (ROM), programmable read-only memory (PROM), electrically erasable programmable read-only memory (EEPROM), random-access memory (RAM), flash memory, disk drive and the like.

Furthermore, the modules, processes, systems, and sections can be implemented as a single processor or as a distributed processor. Further, it should be appreciated that the steps mentioned above may be performed on a single or distributed processor (single and/or multi-core). Also, the processes, modules, and sub-modules described in the various figures of and for embodiments above may be distributed across multiple computers or systems or may be co-located in a single processor or system. Exemplary structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a programmable logic device (PLD), programmable logic array (PLA), field-programmable gate array (FPGA), programmable array logic (PAL) device, or the like. In general, any process capable of implementing the functions or steps described herein can be used to implement embodiments of the method, system, or a computer program product (software program stored on a non-transitory computer readable medium).

Furthermore, embodiments of the disclosed method, system, and computer program product may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a very-large-scale integration (VLSI) design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of control systems, image processing and classification, optical tomography and/or computer programming arts.

Moreover, embodiments of the disclosed method, system, and computer program product can be implemented in software executed on a programmed general purpose computer, a special purpose computer, a microprocessor, or the like.

The foregoing descriptions apply, in some cases, to examples generated in a laboratory, but these examples can be extended to production techniques. For example, where quantities and techniques apply to the laboratory examples, they should not be understood as limiting.

Features of the disclosed embodiments may be combined, rearranged, omitted, etc., within the scope of the invention to produce additional embodiments. Furthermore, certain features may sometimes be used to advantage without a corresponding use of other features.

It is thus apparent that there is provided in accordance with the present disclosure, apparatus, methods, and devices for imaging of a human subject using optical tomography. Many alternatives, modifications, and variations are enabled by the present disclosure. While specific embodiments have been shown and described in detail to illustrate the application of the principles of the present invention, it will be understood that the invention may be embodied otherwise without departing from such principles. Accordingly, Applicants intend to embrace all such alternatives, modifications, equivalents, and variations that are within the spirit and scope of the present invention.

1. An optical tomography device for imaging at least one finger of a patient, the device comprising:
a detection portion with a support configured to permit the hand of a person to rest thereon, the support being attached to a frame for an illuminating device configured to illuminate joint portions of the hand at a plurality of locations thereof;
an imaging portion configured to image light emanating from the hand of the person which results from the illumination at the plurality of the points and to generate image data representative of the light emanating from the hand, the points being surface locations and being at different positions on the surface of the hand;
a processor programmed to receive the image signals and combine the image data representing light emanating from the hand in response to each of the plurality of points;
the processor being further programmed to estimate a distribution of an optical property within the hand by combining the image data corresponding to at least two of the plurality of points.

2. The device of claim 1, wherein the detection portion includes a photodetector or camera configured to detect and discriminate light emanating from each of a variety of points on the surface of the hand.

3. The device of claim 1, wherein the illumination device includes a plurality of waveguides arranged such that each generates a respective one of the points.

4. The device of claim 3, wherein the number of points is at least three.

5-8. (canceled)

9. The device of claim 4, wherein the illumination device includes an arcuate or semi-circular holder for the output ends of the waveguides, the output ends being arranged at substantially equal intervals around the holder, the holder being sized to permit the insertion of a finger between the holder and the support.

10. The device of claim 4, wherein the illuminating device includes an optical switch connected to input ends of the waveguides, and a light source connected to the optical switch, the switch being configured to selectively connect each waveguide to said light source.

11. The device of claim 10, wherein the illuminating device includes a near-IR laser.

12. The device of claim 4, wherein the output ends of the waveguides including collimators or lenses constructed to focus light passing therethrough.

13. The device of claim 12, wherein the arrangement of the output ends with respect to said portion results in a spot size of the light from each waveguide incident on the surface of the portion of less than 1 mm in diameter.

14-16. (canceled)

17. The device of claim 3, further comprising a translation stage configured to move the illumination device to respective positions, each of which is effective to generate a respective one of the plurality of points.

18. (canceled)

19. The device of claim 1, further comprising a plurality of the illumination devices, each of the illumination devices being arranged to illuminate a different finger of the patient's hand.

20-22. (canceled)

23. An optical tomography device for imaging at least one finger of a patient, the device comprising:
a support sized and configured to permit the hand of a human subject to rest thereon;
an illumination device attached to the support;
a surface geometry capture device attached to the support;
a light capture device attached to the support; and
a controller connected to the illumination device, the surface geometry capture device, and the capture device and configured to control each of them;
wherein the illumination device is configured to generate optical sources at multiple locations about a surface of a joint of a hand resting on said support;
wherein the light capture device is configured to provide multiple detectors on a surface about the joint of a hand resting on said support.

24. The device of claim 23, wherein the surface geometry capture device includes at least one camera oriented to image the dorsal surface of a hand.

25. The device of claim 23, wherein the surface geometry capture device includes at least two cameras oriented to image the dorsal surface of a hand from at least two points of view.

26. The device of claim 24, further comprising a positioning device configured to move and/or orient said at least two or said at least one camera, wherein the controller is configured controls said positioning device.

27-28. (canceled)

29. The device of claim 26, wherein said controller is configured to control said illumination device to generate multiple sources located at multiple locations along a longitudinal axis of the finger.

30-31. (canceled)

32. The device of claim 23, wherein the light capture device includes a CCD camera configured to provide multiple detectors on a surface about the joints of multiple fingers resting on said support.

33. The device of claim 32, wherein said controller is configured to control said illumination device to generate multiple sources located at multiple locations along a lateral and/or longitudinal axis of each of the multiple fingers.

34. (canceled)

35. A method for acquiring a cross-sectional image of a body part of a human subject, the method comprising:
(a) illuminating multiple points on a surface of the body part at a desired cross-sectional plane;
(b) detecting light from the body part as a result of the illuminating in (a);
(c) illuminating another point on the surface of the body part at the desired cross-sectional plane;
(d) detecting light from the body part as a result of the illuminating in (c); and
(e) generating the cross-sectional image of the body part of the human subject based on the light detecting in (b) and (d).

36. The method of claim 35, further comprising repeating (a) through (e) at a different cross-sectional plane.

37. The method of claim 35, further comprising repeating (a) through (e) for multiple different cross-sectional planes, and using the cross-sectional images to generate a three-dimensional cross-section of the body part.

38. The method of claim 36, wherein the repeating includes translating a light source that provides the illuminating to a different location.

39. An optical tomography device for imaging at least one finger of a patient, the device comprising:
a support including an enclosure cover sized and configured to enclose the hand or finger of a human subject, the enclosure having a lining defining an inwardly-facing surface arranged to face, and contact, an enclosed hand, the sources and detectors being arrayed on the inwardly-facing surface;
light sources and detectors attached to the surface; and
a controller connected to the sources and detectors and configured to control them to generate an optical tomographic image;
the sources and detectors are arranged to generate optical sources at multiple locations and optical detectors at multiple further locations about a surface of a joint of a hand of finger adjacent said surface;
wherein the light capture device is configured to provide multiple detectors on a surface about the joint of a hand or finger resting on said support.

40. The device of claim 39, wherein the lining includes sensor elements configured to output a signal indicating at least one geometric parameter of said surface.

41. The device of claim 40, wherein said sensor elements include capacitors.

42. The device of claim 40, wherein said sensor elements include strain gauges.

43. The device of claim 42, wherein the support includes a finger cot with the sources and detectors supported thereby.

44. (canceled)

45. The device of claim 39, wherein the enclosure cover includes a rigid enclosure element that supports the lining which together encase a resilient foam member which resiliently supports the lining.

46-49. (canceled)

50. The device of claim 39, wherein the enclosure is configured to clip onto a joint.

51-54. (canceled)

55. The device of claim 39, wherein the enclosure is shaped as a glove without at least one of the fingers thereof being closed at a fingertip thereof.

56-75. (canceled)
